(19) United States
(12) Patent Application Publication
(10) Pub. No.: US 2010/0268174 A1
(43) Pub. Date: Oct. 21, 2010

(54) COMBINATION OF ALCOHOL LOCK AND GENTIAN VIOLET CATHETER

(76) Inventors: Elaine Martha Steinke, Myerstown, PA (US); Daniel J. Spangler, Fort Worth, TX (US); Joel Rosenblatt, Pottstown, PA (US)

Correspondence Address: AMSTER, ROTHSTEIN & EBENSTEIN LLP, 90 PARK AVENUE, NEW YORK, NY, 10016, US

(21) Appl. No.: 12/733,992

(22) PCT Filed: Oct. 2, 2008
(86) PCT No.: PCT/US08/11389

§ 371 Date: Jun. 15, 2010

Related U.S. Application Data
(60) Provisional application No. 60/997,403, filed on Oct. 3, 2007.

(51) Int. Cl. A61L 15/46

(52) U.S. Cl. 604/265

(57) ABSTRACT

Implantable catheters treated with gentian violet and methods for disinfecting the catheters with alcohol are provided.

### CROSS-REFERENCE TO RELATED APPLICATION

This application claims the benefit of U.S. Provisional Patent Application No. 60/997,403, filed on Oct. 3, 2007, the content of which is hereby incorporated by reference into the subject application.

### FIELD OF THE INVENTION

The present invention relates to medical devices, especially catheters such as intravenous catheters, with gentian violet incorporated in the device either as a coating or bulk distributed, used in synergistic combination with an alcohol lock solution to inhibit attachment and/or growth of microorganisms, thereby preventing device-related infection.

### BACKGROUND OF THE INVENTION

Various publications are referred to throughout this application. Full citations for these references may be found at the end of the specification immediately preceding the claims. The disclosures of these publications are hereby incorporated by reference in their entireties into the subject application to more fully describe the art to which the subject application pertains.

Intravenous catheters, when placed in the human body, serve as an attachment point for microorganisms, leading to biofilm formation and infection. Infection of the catheter hub and catheter-related blood stream infections are major complications for patients with indwelling catheters (e.g., Safdar and Maki 2003; Saint et al. 2000).

The antimicrobial activity of ethyl alcohol (ethanol) as well as other alcohols is well known. Isopropyl alcohol at a concentration of 60-70% is widely used as an antimicrobial agent for sanitization of surfaces and skin. A concentration of 10% ethyl alcohol inhibits the growth of most microorganisms, while concentrations of 40% and higher are generally considered bactericidal (Sissons et al. 1996).

Antimicrobial lock solutions have been used to address luminal introduction of microorganisms to patients' blood stream. The use of ethanol as a lock solution is known (Ball et al. 2003; Dannenberg et al. 2003; Metcalf et al. 2004; University of Wisconsin News Release, Aug. 10, 2005, U.S. Pat. No. 6,350,251, U.S. Patent Application Publication Nos. US 2005/0013836 and US 2007/0202177). Polymers commonly utilized to produce intravascular devices have been shown to be compatible with 70% ethyl alcohol (Crnich et al. 2005); however, not all polymers are compatible. The addition of other antimicrobial agents to lock solutions of lower alcohols, including ethyl alcohol, has also been described (U.S. Patent Application Publication Nos. US 2005/0013836 and US 2007/0202177). A catheter hub containing an antiseptic chamber filled with 3% iodinated alcohol has been shown to significantly reduce the rate of catheter-related blood stream infections, when compared with a standard hub model (Segura et al. 1996). Finch et al. disclose in U.S. Pat. Nos. 6,592,564, 6,679,870 and 6,685,694 the addition of triclosan or taurolidine to catheter lock solutions of lower alcohols, including ethyl alcohol (ethanol). The addition of antibiotics to catheter lock solutions has been described as an infection prevention (prophylaxis) and treatment approach (Bestul et al. 2005; O'Grady et al. 2002).

One approach to help prevent device-related infection is to treat the device with the antiseptic gentian violet (GV). U.S. Pat. No. 5,709,672 describes a solvent impregnation method of gentian violet. U.S. Patent Application publications 2003/0078242 A1, 2005/0131356 A1 and 2005/0197634 A1 also teach solvent impregnation for dyes such as gentian violet. Hanna et al. (2006) describe impregnation of catheters with gendine, which contains gentian violet and chlorhexidine. Antimicrobial polymer compositions containing gentian violet that can be used to coat medical devices have been described (PCT International Publication No. WO 03/066721). Shanbrom (U.S. Pat. No. 6,361,786) describes the use of antimicrobial dyes such as gentian violet in combination with meglumine on other medical devices. U.S. Patent Application Publication No 2007/0129690 describes a catheter with an antimicrobial agent such as gentian violet disposed at the proximal portion of the main body of the catheter.

Despite these advances in combating infection, improved approaches are needed to prevent infections of implantable medical devices such as catheters. Prevention of catheter related blood stream infection, particularly with chronic catheters, remains a significant need.

### SUMMARY OF THE INVENTION

The present invention is based on the discovery of a synergistic relationship between gentian violet and alcohol to combat infection of implantable medical devices such as catheters with reduced risk of toxicity.

The invention provides implantable catheters comprising a lumen that is at least partly filled with a solution comprising alcohol, wherein the catheter is impregnated with gentian violet and/or the inner surface of the lumen is coated with gentian violet and wherein gentian violet is present in the catheter in a concentration of 0.01%-20% by weight of the catheter.

The invention also provides implantable catheters that can be disinfected in vivo with alcohol, wherein the catheter is impregnated with gentian violet and/or the catheter comprises an inner lumen that is coated with gentian violet, and wherein gentian violet is present in the catheter in a concentration that is subinhibitory for attachment or growth of microorganisms on the catheter.

The invention further provides methods of disinfecting or preventing infection of a catheter implanted in a subject, the method comprising flushing the inner lumen of the catheter with a solution comprising alcohol, where the implanted catheter is impregnated with gentian violet and/or the inner lumen is coated with gentian violet and wherein gentian violet is present in the catheter in a concentration of 0.01%-20% by weight of the catheter.

### DETAILED DESCRIPTION OF THE INVENTION

The invention provides an implantable catheter comprising a lumen that is at least partly filled with a solution comprising alcohol, wherein at least an inner surface of the lumen is impregnated or coated with gentian violet and wherein gentian violet is present in a concentration of 0.01%-20% by weight of the catheter.

In different examples of the catheter, gentian violet is present in a concentration of at least 0.05%, or at least 0.1%, or at least 0.5%, or at least 1% by weight of the catheter. Gentian violet can present in a concentration of at least 15 μg gentian violet per cm² area of the catheter, for example, in a concentration of 50-500 μg gentian violet per cm² area of the catheter.

The gentian violet can be present in the catheter in a concentration that, in the absence of alcohol, is subinhibitory for attachment or growth of microorganisms on the catheter, and/or alcohol can be present in a concentration that, in the absence of gentian violet, is subinhibitory for attachment or growth of microorganisms on the catheter. As used herein, a “subinhibitory” concentration of gentian violet or alcohol is a concentration that is below the concentration required to prevent or reduce attachment or growth of microorganisms on an implantable catheter. Minimum inhibitory concentration can be determined, for example, as set forth herein in the Examples.

The alcohol can be, for example, one or more of ethanol, propanol, isopropanol and butanol. Ethanol is a preferred alcohol. The solution containing alcohol can further comprise an anti-coagulant, such as, for example, heparin. The alcohol can be present in the solution in a concentration of, for example, 25-30%.

The invention also provides an implantable catheter that can be disinfected in vivo with alcohol, wherein the catheter comprises a lumen with an inner surface that is impregnated or coated with gentian violet and wherein gentian violet is present in the catheter in a concentration that is subinhibitory for attachment or growth of microorganisms on the catheter. The catheter can be disinfected in vivo with a solution comprising alcohol at a concentration that, in the absence of gentian violet, is subinhibitory for attachment or growth of microorganisms on the catheter.

When the implantable catheter is implanted in a subject and not in use, the catheter can be filled with a solution containing alcohol and then capped off or “locked.” The alcohol lock can contain one or more anticoagulants. The alcohol lock can be left in place as long as the catheter is not in use or for a period of time specified by a physician.

The invention provides a method of disinfecting or preventing infection of a catheter implanted in a subject, the method comprising flushing a lumen of the catheter with a solution comprising alcohol, wherein at least an inner surface of the lumen is impregnated or coated with gentian violet and wherein gentian violet is present in a concentration of 0.01%-20% by weight of the catheter. In different examples of the catheter, gentian violet is present in a concentration of at least 0.05%, or at least 0.1%, or at least 0.5%, or at least 1% by weight of the catheter. Gentian violet can present in a concentration of at least 15 μg gentian violet per cm² area of the catheter, for example, in a concentration of 50-500 μg gentian violet per cm² area of the catheter.

In the methods of the present invention, gentian violet can be present in the catheter in a concentration that, in the absence of alcohol, is subinhibitory for attachment or growth of microorganisms on the catheter, and/or alcohol can be present in a concentration that, in the absence of gentian violet, is subinhibitory for attachment or growth of microorganisms on the catheter.

The catheter can be disinfected in vivo with a solution comprising 5-100% alcohol, preferably 25-70% alcohol, more preferably 25-50% alcohol, and still more preferably 25-30% alcohol. The alcohol can be, for example, one or more of ethanol, propanol, isopropanol and butanol. Ethanol is a preferred alcohol. The solution containing alcohol can further comprise an anti-coagulant, such as, for example, heparin or citrate.

Preferably, with the implantable catheters and methods of the present invention, growth of one or more of fungal, gram positive or gram negative pathogenic microorganisms including Candida albicans, Staphylococcus aureus, or Pseudomonas aeruginosa is inhibited. The radius of the zone of inhibition (ZOI) of microorganisms, such as e.g. Candida albicans, Staphylococcus aureus or Pseudomonas aeruginosa, around the catheter can increased by at least 25% in the presence of 5% ethanol, compared to the radius of the ZOI in the absence of ethanol. Preferably, the radius of the zone of inhibition (ZOI) of microorganisms, such as e.g. Candida albicans or Pseudomonas aeruginosa, around the catheter is increased by at least 50%, or by at least 75%, in the presence of 5% ethanol, compared to the radius of the ZOI in the absence of ethanol. More preferably, the radius of the zone of inhibition (ZOI) of microorganisms, such as e.g. Pseudomonas aeruginosa, around the catheter is at least doubled in the presence of 5% ethanol, compared to the radius of the ZOI in the absence of ethanol. The zone of inhibition can be determined, for example, as set

Gentian violet can be the only anti-infective agent impregnated in or coated on the catheter. However, in addition to gentian violet, the catheter can also be impregnated with or coated with a substance selected from the group consisting of one or more of antibiotics, minocycline, rifampin, clindamycin, antifungals, antiseptics, antimicrobial dyes, biguanides, phenolics, quarternary ammonium salts, cationic steroids, triclosan, ethylene diamine tetraacetic acid (EDTA), citrate, taurolidine, 5-fluorouracil, silver, silver salts, miconazole, ketoconazole, chlorohexidine or itraconazole or their derivatives or salts.

The chlorhexidine in the catheter can be in the form of chlorhexidine base and/or a chlorhexidine salt. Chlorhexidine salts include, for example, chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-alpha-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynaphthoate, and chlorhexidine embonate. Preferred forms of chlorhexidine include chlorhexidine base and chlorhexidine succinate.

The catheter can be implanted in a subject, for example in a vessel such as a blood vessel or in a body cavity. Examples of such catheters include transcutaneous catheters; vascular catheters including peripheral catheters, central catheters, venous catheters, and arterial catheters; urinary catheters; and dialysis catheters.

The present invention is illustrated in the following Experimental Details section, which is set forth to aid in the understanding of the invention, and should not be construed to limit in any way the scope of the invention as defined in the claims that follow thereafter.

### EXPERIMENTAL DETAILS

### Example I

### Inhibition of Growth of Microorganisms Using Gentian Violet Treated Catheters and Alcohol

Determinations of Minimum Inhibitory Concentrations (MIC) and Minimum Bacteriocidal Concentrations (MBC) for ethanol against several pathogens of interest were performed using standard microbiological techniques. Ethanol was tested at the following percent (%) concentrations: 50, 40, 30, 25, 20, 15, 12, 10, 7 and 5. Dilutions were prepared in Cationic Adjusted Mueller Hinton Broth (CAMHB). All tests were performed in microtiter plates with a total test volume of 100 μL, including diluted antimicrobial agent and inoculum. Concentrations of antimicrobial agents were prepared at 2× concentration, and 50 μL was added to each well of a microtiter plate, to which 50 μL of inoculum of the test organism was added to give the target test concentration.

Inoculum was prepared by transfer of isolated colonies from agar plates to 10 mL of trypticase soy broth (TSB), and incubation at 37° C. for 4 hours. Cells were subsequently centrifuged, and resuspended twice in phosphate buffered saline. The population of this bacterial suspension was determined by reading optical density at 670 nm. Final concentrations of inoculum were adjusted to a range of approximately 5×10⁶ cfu/mL, using CAMHB as a diluent. Initial concentrations were verified using the Miles and Misra drop count method. To each test well of a microtiter plate, 50 μl of inoculum was added to each test well along with 50 μl of the 2× concentration of the antimicrobial agent being tested. Incubation was at 37° C. for 16 to 20 hours.

The MIC was the lowest concentration of antimicrobial agent that completely inhibited growth of the organism in the micro dilution wells as detected by the unaided eye. To determine MBC values, 50 μl was removed from each well showing inhibition of growth (MIC and greater). Solutions from these inhibitory concentrations of antimicrobial agent were diluted in CAMHB to concentrations where they would no longer inhibit the growth of the test organism. Recovery counts were obtained by plating aliquots of the diluted material onto TSA plate containing 5% sheep's blood, and incubating 24 hours at 37° C. The MBC value was calculated as the concentration where the colony forming units equal 1% of those found at the MIC.

Minimum Inhibitory (MIC) and Minimum Bacteriocidal (MBC) ethanol concentrations measured for a variety of common pathogens values are reported in Table 1. These results suggest a lock at ethanol concentrations above 25% will be cidal towards most commonly encountered infectious pathogens. At concentrations below 7% the solution will not inhibit microbial growth. 5% ethanol was determined to be a subinhibitory concentration for all organisms of interest.

TABLE 1
Minimum Inhibitory Concentration (MIC) and Minimum Bacteriocidal
Concentration (MBC) for ethanol for a variety of common pathogens.

| Organism | MIC | MBC |
|---|---|---|
| P. aeruginosa ATCC 27853 | 7% | 12% |
| E. cloacae ATCC 13047 | 7% | 10% |
| E. faecalis ATCC 51299 | 7% | 10% |
| C. albicans ATCC 10231 | 7% | 10% |
| K. pneumoniae ATCC 10031 | 10% | 25% |
| E. coli ATCC 25922 | 10% | 15% |
| S. aureus ATCC 29213 | 12% | 25% |
| S. epidermidis ATCC 35983 | 12% | 10-12% |

ATCC, American Type Culture Collection, Manassas, Virginia.

To test the combination of gentian violet treated catheters at subinhibitory ethanol concentrations, 15 French polyurethane (Tecothane) catheters were treated for 2 hours with 1%, 0.5%, 0.1%, 0.05%, and 0.01% gentian violet solutions (solvent 80/15/5 methylethylketone/water/acetone). The catheters were then air dried for 2 hours followed by vacuum drying at 50° C. overnight. Dried catheter segments were cut and inserted into Mueller-Hinton 2 agar (MH) either with ethanol added to a final dilution of 5% or without any added ethanol. Plates were incubated at 37° C. and zones of inhibition were measured after 24 hours.

As indicated in Table 2, the use of subinhibitory ethanol concentrations in combination with the gentian violet treated catheter provides significant enhancement in antimicrobial protection over the use of either treatment alone.

TABLE 2
Zone of Inhibition (ZOI) (mm) of Gentian Violet treated catheters in
Mueller-Hinton Agar with and without 5% ethanol.

| | Gentian violet treatment (ZOI in mm) | | | | |
|---|---|---|---|---|---|
| Organism/media | 1% | 0.5% | 0.1% | 0.05% | 0.01% |
| CA-MH | 28.9 | 27.3 | 24.24 | 19.7 | 11.0 |
| CA-MH & | 54.5 | 51.0 | 41.2 | 35.65 | 21.2 |
| 5% EtOH | | | | | |
| PA-MH | 10.5 | 9.7 | 0 | 0 | 0 |
| PA-MH & | 22.6 | 22.2 | 15.6 | 7.9 | 5.8 |
| 5% EtOH | | | | | |
| SA-MH | 21.6 | 21.1 | 16.1 | 13.1 | 6.5 |
| SA-MH & | 24.3 | 23.8 | 18.5 | 16.8 | 8.9 |
| 5% EtOH | | | | | |

CA denotes Candida albicans;
SA denotes Staphylococcus aureus;
PA denoted Pseudomonas aeruginosa;
MH denotes Mueller-Hinton 2 agar;
MH & 5% EtOH denotes Mueller-Hinton 2 agar containing 5% ethanol.

### Examples II-VI

### Provide Examples of Gentian Violet Treated Catheters

### Examples II-III

### Incorporation of Gentian Violet into Tecothane®-2095A Resin

Experiments were performed to incorporate gentian violet into Tecothane®-2095A resin by compounding and extrusion processes. In these examples gentian violet was coated on Tecothane®-2095A pellets by soaking the resin in gentian violet/ethanol mixture and the solvent was evaporated off at ambient conditions. The gentian violet coated pellets were then fed into an extruder or compounder to make tubing or strand pellitized pellets. The gentian violet could also have been fed as a powder directly with the polymer resin for compounding and extrusion. Surprisingly, much higher loadings of gentian violet could be achieved using the high temperature process disclosed herein than had been previously disclosed without degradation of the chemical structure of the gentian violet.

### Example 2

### Compound Tecothane®-2095A Resin with 0.5% Gentian Violet

5 g of gentian violet (Sciencelab, Houston, Tex.) was dissolved in 250 ml 99% ethanol (Sigma-Aldrich, St. Louis, Mo.). 1000 g of Tecothane® 2095A (Noveon, Cleveland, Ohio) resin was added in to the gentian violet/ethanol solution. The ethanol solvent was evaporated off in a chemical fume hood overnight at ambient conditions. The gentian violet coated pellets were then dried at 50° C. and 30 inches Hg for 24 hrs prior to compounding.

The dried gentian violet coated resins were starve-fed into a 18 mm Leistritz intermeshing twin screw extruder (Somerville, N.J.) from a K-iron feeder (Pitman, N.J.) at a rate of 2.5 kg/hr. The extruder was set at 231 rpm for screw speed and the barrel zone temperatures were set from 329° F. (165° C.) thru 338° F. (170° C.). The extrudate was pelletized into small pellets.

### Example 3

### 2% Gentian Violet Loaded Tecothane® Tube

20 g of gentian violet (Sigma-Aldrich, St. Louis, Mo.) was dissolved in 1000 ml ethanol (Sigma-Aldrich, St. Louis, Mo.). 1000 g of Tecothane® resin was added into the gentian violet/ethanol solution. The ethanol solvent was evaporated off in the chemical fume hood overnight at ambient conditions. The gentian violet coated resin then dried at 65° C. and 30 inches Hg for 4 hrs prior to compounding.

The dried gentian violet coated resins were gravity fed into a 5/8′ Randcastle single screw (Cedar Grove, N.J.) microextruder. The microextruder was set at 20 rpm for screw speed and barrel zone temperatures were set from 360° F. thru 375° F. A 5 Fr tubing was drawn from a BH25 tooling (San Marcos, Calif.).

### Characterization of Gentian Violet Compounded in Tecothane® Via HPLC

Gentian violet contents from compounded resin and tube sample were analyzed via HPLC method. HPLC analysis on GV loaded resin or compounded pellets was performed by weighing 0.1-0.3 g, which is roughly equivalent to 10-20 pellets (depending on the weight of the raw material used) of each formulation (n=3) and digesting in THF (5 mL or 7.5 mL) in 50 mL centrifuge tubes. Samples were allowed to sit for 45 minutes, and then vortexed until all of the polymer dissolved. An equal amount of deionized (DI) water was then added (5 or 7.5 mL), and the samples were again vortexed for 5 minutes, and then centrifuged for 10 minutes. A portion of each sample was then added to an HPLC vial and capped prior to HPLC analysis.

HPLC analysis on tubing was performed by cutting and measuring 1 cm segments of each formulation (n=3) and digesting in THF (10 mL or 20 mL) in 50 mL centrifuge tubes. Samples were allowed to sit for 45 minutes, and then vortexed for 5 minutes, or until none of the polymer was stuck to the bottom of the tubes. An equal amount of deionized (DI) water was then added (10 or 20 mL), and the samples were again vortexed for 5 minutes, and then centrifuged for 10 minutes. A portion of each sample was then added to an HPLC vial and capped prior to HPLC analysis.

HPLC analysis was performed on an Agilent 1200 Series LC using an Agilent Eclipse XDB-CN 5μ 4.6×150 mm column with the corresponding guard column. A gradient program was run using two solvent reservoirs:

MP A: 100% DI Water/0.2% Trifluoroacetic Acid,

MP B: 100% Acetonitrile/0.2% Trifluoroacetic Acid.

### Program: 0-5 min 35% B, 5-6 min 35-40% B, 6-12 min 40% B, 12-16 min 35% B.

The gentian violet content of the compounded resin and extruded tube is shown in Table 3. Table 4 shows a comparison of the relative peak areas of gentian violet peaks of standard vs. sample. Percent area is based only on the total area of the three peaks, and not any other peaks in the chromatograms. Table 5 shows a comparison of the relative peak areas of gentian violet peaks of standard vs. sample (from Aldrich).

TABLE 3
GV content of compounded resin and extruded tube.

| | Theoretical | Measured GV loading |
|---|---|---|
| Sample | loading (% w/w) | (% w/w) via HPLC |
| Compounded | 0.5 | 0.67% |
| Resin | | |
| Tube | 2.0 | 2.2% |

TABLE 4
Relative % Areas of each peak, Standard vs Compounded sample.

| | |
|---|---|
| Standard | Sample |

| | | Relative | | Relative % |
|---|---|---|---|---|
| Peak # | Area (uV−s) | % area | Area (uV−s) | area |
| Peak 1 | 640917.67 | 14.75% | 191846.97 | 14.22% |
| Peak 2 | 1789022.3 | 41.17% | 583737.5 | 43.28% |
| Peak 3 | 1915778.8 | 44.08% | 573305.43 | 42.50% |
| Total area | 4345718.8 | | 1348889.9 | |

TABLE 5
Relative % Peak Area, Standard vs tube sample.

| | % Relative Peak area | | |
|---|---|---|---|
| | Peak 1 | Peak 2 | Peak 3 |
| Tube sample | 1.51 | 18.27 | 80.21 |
| Standard | 1.93 | 19.47 | 78.59 |

### Example IV

### Temperature Effect on Degradation of Extruded Gentian Violet. Gentian Violet in Tecothane® Extrusions w/30% Filler

Extruded gentian violet was found to degrade at a processing temperature of 217° C. (starting at about 209° C.); however, processing under about 207° C. does not degrade gentian violet. One of the surprising findings about the temperature effects is that certain classes of polyurethanes are processable below the degradation temperature and other classes are not.

Compounding Conditions for Stable Gentian Violet:

Barrel Temperatures:
Zone 1-5=170° C.
Zone 6=175° C.,

Melt Temp=194° C.,

Screw Speed=100 RPM,

Pressure=˜180 bar.

Compounding Temperature for Degraded Gentian Violet:

Zone 1-6=217° C.

Compounding was performed with Sigma Aldrich ACS grade gentian violet, lot 016K3691. HPLC analysis was performed by cutting and measuring 1 cm segments of each formulation (n=3) using the method described in Examples II and III.

The chromatograms show the stable gentian violet followed by the degraded gentian violet, at 588 nm, 253 nm, and 280 nm. The degradation peaks from the 217° C. sample are evident as the new peaks at 253 and 280 nm wavelengths. The chromatogram of the low processing temperature was identical to that of the Gentian Violet raw material.

### Example V

### Temperature Effect (196° C.-219° C.) on Degradation of Extruded Gentian Violet. Compounded Carbothane® resin with 0.5% GV.

5 g of gentian violet (Sigma-Aldrich, St. Louis, Mo.) was dissolved in 250 ml 99% ethanol (Sigma-Aldrich, St. Louis, Mo.). 1000 g of Carbothane®-3585A-B20 (Noveon, Cleveland, Ohio) resin was added in to the gentian violet/ethanol solution. The ethanol solvent was evaporated off in a chemical fume hood overnight at ambient conditions. The gentian violet coated pellets were then dried at 50° C. and 30 inches Hg for 24 hrs prior to compounding.

The dried gentian violet coated resins were starve-fed into a 18 mm Leistritz intermeshing twin screw extruder (Somerville, N.J.) from a K-Tron feeder (Pitman, N.J.) at a rate of 2.5 kg/hr. The heating profile for each run was varied. The melt temperatures from each run are recorded in Table 6.

HPLC analysis was performed by selecting several pellets of each formulation (n=3) using the method described in Examples II and III.

GV content is essentially unchanged up to a processing temperature of 206° C. The onset of degradation appears at 209° C. and becomes worse at 219° C.

TABLE 6
GV Contents from compounded Carbothane ®
at moderate temperatures.

| | | | GV measured from |
|---|---|---|---|
| | | GV measured from | compounded resin |
| | Melt Temp | unprocessed resin | (% w/w) |
| Sample # | (° C.) | (% w/w) | via HPLC |
| GV control | | 0.50 | |
| 3 | 196 | | 0.49 |
| 4 | 206 | | 0.51 |
| 5 | 209 | | 0.43 |
| 6 | 219 | | 0.40 |

### Example VI

### Temperature Effect on Degradation of Extruded Gentian Violet—High Temperatures. Compounded Carbothane® Resin with 0.5% GV

The sample preparation for loading GV on resin pellets is similar to EXAMPLE V. Gentian violet was purchased from (Sigma-Aldrich, St. Louis, Mo.) and Carbothane®-3585A-B20 (Noveon, Cleveland, Ohio) was used. The heating profile was varied for each run. The melt temperatures are showed in Table 7.

There is significant degradation of the GV above 220° C. as evidenced by the dramatic drop in extractable GV content.

TABLE 7
GV contents from compounded Carbothane ®
at high temperatures.

| | | GV measured from | GV measured from |
|---|---|---|---|
| | Melt Temp | unprocessed resin | compounded resin (% |
| Sample # | (° C.) | (% w/w) | w/w) via HPLC |
| GV control | | 0.45 | |
| 7 | 188 | | 0.42 |
| 8 | 221 | | 0.15 |
| 9 | 230 | | 0..13 |
| 10 | 235 | | 0.11 |
| 11 | 241 | | 0.09 |
| 12 | 246 | | 0.06 |

### Discussion

The present invention relates to medical devices, preferably intravenous catheters, with gentian violet incorporated in the device either as a coating or bulk distributed used in synergistic combination with an alcohol lock solution to inhibit attachment and/or growth of microorganisms, thereby preventing device-related infection. The addition of gentian violet directly into the lock solution can be undesirable in that it is a dye and can stain the catheter extension lines as well as the patient and care providers' clothing and skin during infusion and aspiration of the locking solution. In addition, the risk of inadvertent flushing of the lock into the patient's bloodstream can expose the patient to potentially toxic side effects. These include enhanced local irritation and necrosis over ethanol alone. Since the microbial attachment and colonization associated with catheter related bloodstream infections occurs at the surface of the device, the inventors have surprisingly found that sufficiently high surface concentrations of gentian violet and ethanol can be attained such that they are bactericidal against common infectious pathogens with reduced risk of toxicity and staining. Sublethal ethanol concentrations can occur at the tip of the catheter by leakage or diffusion of ethanol into the blood stream. The inventors have surprisingly found that even if the ethanol concentration drops to just 5% (a sublethal ethanol level), gentian violet potency is increased substantially. If gentian violet were directly dissolved in the lock solution, leakage and diffusion of both it and ethanol from the catheter tip could result in local levels (at the tip) insufficient to kill potential infectious pathogens. Surprisingly, gentian violet can be beneficially released from the surface of the catheter at sufficiently high local concentrations to substantially enhance the potency of the combination (even at sublethal ethanol concentrations) against highly recalcitrant pathogens such as Candida yeast species.

This invention allows the effective use of lower ethanol concentrations in locks. Clinical usage of ethanol lock solutions has ranged from concentrations of 25% to 100%. More effective use of ethanol concentrations at the low end of this range (towards 25%) enhances safety to the patient from two standpoints. One is the previously mentioned reduced risk of toxic side effects due to exposure of tissues to high local ethanol concentrations, and the second is reduced risk of compromise of catheter mechanical strength due to prolonged exposure of catheter materials to higher ethanol concentrations. In addition to gentian violet, other antimicrobial agents (such as other antiseptic dyes, antiseptics, antibiotics, chemotherapeutics, antimycotics or combinations thereof) can be incorporated into catheters to enhance rates of inhibition and lethality towards pathogens at catheter surfaces in combination with an ethanol lock. Of special note are the antimicrobial agents antibiotics, antifungals, antiseptics, minocycline (or other tetracyclines), rifampin, clindamycin, triclosan, ethylene diamine tetraacetic acid (EDTA), citrate, silver, silver salts, taurolidine, 5-fluorouracil, miconazole, ketoconazole, chlorohexadine and itraconazole.

### REFERENCES

Ball P A, Brokenshire E, Parry B, Merrie A, Gillanders L, Mcllroy K, Plank L. Ethanol locking as a possible treatment for microbial contamination of long-term central venous catheters. Nutrition 2003; 19:570.
Bestul et al. Antibiotic Lock Technique: Review of the Literature. Pharmacotherapy. 2005; 25 (2):211-227.
Crnich C J, Halfmann J A, Crone W C, Maki D G. The effects of prolonged ethanol exposure on the mechanical properties of polyurethane and silicone catheters used for intravascular access. Infect Control Hosp Epidemiol. 2005 August; 26(8):708-14.
Dannenberg C, Bierbach U, Rothe A, Beer J, Korholz D. Ethanol-lock technique in the treatment of bloodstream infections in pediatric oncology patients with brovaic catheter. J Pediatr Hematol Oncol 2003; 25(8):616-21.
Hanna H, P Bahna, R Reitzel, T Dvorak, G Chaiban, R Hachem and I Raad. Comparative in vitro efficacies and antimicrobial durabilities of novel antimicrobial central venous catheters. Antimicrob. Agents Chemother. 50: 3283-3288, 2006.
Mecalf S C, Chambers S T, Pithie A D. Use of ethanol locks to prevent recurrent central line sepsis. J. Infect. 2004; 49:20-2.
O'Grady et al. Guidelines for the Prevention of Intravascular Catheter-Related infections. Clinical Infectious Diseases 2002; 35:1281-1307.

Safdar N, Maki D G. The pathogenesis of catheter-related bloodstream infection with noncuffed short-term central venous catheters. Intensive Care Med. 2004 January; 30(1):62-7. Epub 2003 November 26.
Saint S, Veenstra D L, Lipsky B A. The clinical and economic consequences of nosocomial central venous catheter-related infection: are antimicrobial catheters useful? Infect Control Hosp Epidemiol. 2000 June; 21(6):375-80.
Segura et al. A clinical trial on the prevention of catheter-related sepsis using a new hub model. Ann Surg. 1996; 223:363-369.
Sissons et al. Inhibition by ethanol of the growth of biofilm and dispersed microcosm dental plaques. Archives of Oral Biology, Vol. 41, 1, JN 1996; 27-34.
University of Wisconsin News Release. Ethanol Treatment May Be Instrumental in Fighting IV-Based Infection, http:www.news.wisc.edu/releases/11399.html, Aug. 10, 2005.
PCT International Publication No. WO 03/066721 A1, published Aug. 14, 2003, Michigan Biotechnology Institute, Antimicrobial polymer.
U.S. Patent Application Publication No. US 2003/0078242 A1, published Apr. 24, 2003, Raad et al., Novel antiseptic derivatives with broad spectrum antimicrobial activity for the impregnation of surfaces.
U.S. Patent Application Publication No. US 2005/0013836 A1, published Jan. 20, 2005, Raad, Antimicrobial flush solutions.
U.S. Patent Application Publication No. US 2005/0131356 A1, published Jun. 16, 2005, Ash et al. Medical devices exhibiting antibacterial properties.
U.S. Patent Application Publication No. US 2005/0197634A1, published Sep. 8, 2005, Raad et al., Methods for coating and impregnating medical devices with antiseptic compositions.
U.S. Patent Application Publication No 2007/0129690, published Jun. 7, 2007, Rosenblatt et al., Catheter with polymeric coating.
U.S. Patent Application Publication No. 2007/0202177, published Aug. 30, 2007, Hoang, Antimicrobial Compositions and Methods for Locking Catheters.
U.S. Pat. No. 5,709,672, issued Jan. 20, 1998, Illner, Silastic and polymer-based catheters with improved antimicrobial/antifungal properties.
U.S. Pat. No. 6,350,251, issued Feb. 26, 2002, Prosl et al. Biocidal locks.
U.S. Pat. No. 6,361,786, issued Mar. 26, 2002, Shanbrom, Microbicide treated polymeric materials.
U.S. Pat. No. 6,592,564 B2, issued Jul. 15, 2003, Finch et al., Methods and kits for locking and disinfecting implanted catheters.
U.S. Pat. No. 6,679,870 B1, issued Jan. 20, 2004, Finch et al., Methods and kits for locking and disinfecting implanted catheters.
U.S. Pat. No. 6,685,694 B2, issued Feb. 3, 2004, Finch et al., Methods and kits for locking and disinfecting implanted catheters.

1. An implantable catheter comprising a lumen that is at least partly filled with a solution comprising alcohol, wherein at least an inner surface of the lumen is impregnated or coated with gentian violet and wherein gentian violet is present in a concentration of 0.01%-20% by weight of the catheter.

2-8. (canceled)

9. The implantable catheter of claim 1, wherein gentian violet is present in a concentration of 50-500 μg gentian violet per cm² area of the catheter.

10. The implantable catheter of claim 1, wherein gentian violet is present in a concentration that, in the absence of alcohol, is subinhibitory for attachment or growth of microorganisms on the catheter.

11. The implantable catheter of claim 1, wherein alcohol is present in a concentration that, in the absence of gentian violet, is subinhibitory for attachment or growth of microorganisms on the catheter.

12. The implantable catheter of claim 1, wherein gentian violet is present in a concentration that, in the absence of alcohol, is subinhibitory for attachment or growth of microorganisms on the catheter, and wherein alcohol is present in a concentration that, in the absence of gentian violet, is subinhibitory for attachment or growth of microorganisms on the catheter.

13. The implantable catheter of claim 1, wherein alcohol is present in the solution in a concentration of 25-30%.

14. The implantable catheter of claim 1, wherein growth of Candida albicans, Staphylococcus aureus, or Pseudomonas aeruginosa is inhibited.

15-24. (canceled)

25. The implantable catheter of claim 1, wherein the solution comprises an anti-coagulant.

26. The implantable catheter of claim 1, wherein the catheter is implanted in a subject.

27-28. (canceled)

29. An implantable catheter that can be disinfected in vivo with alcohol, wherein the catheter comprises a lumen with an inner surface that is impregnated or coated with gentian violet and wherein gentian violet is present in the catheter in a concentration that is subinhibitory for attachment or growth of microorganisms on the catheter.

30-31. (canceled)

32. The implantable catheter of claim 29, wherein the radius of the zone of inhibition (ZOI) of microorganisms around the catheter is at least doubled in the presence of 5% ethanol, compared to the radius of the ZOI in the absence of ethanol.

33. (canceled)

34. The implantable catheter of claim 29, wherein the catheter can be disinfected in vivo with a solution comprising alcohol at a concentration that, in the absence of gentian violet, is subinhibitory for attachment or growth of microorganisms on the catheter.

35-37. (canceled)

38. The implantable catheter of claim 29, wherein the catheter is implanted in a subject.

39-40. (canceled)

41. A method of disinfecting or preventing infection of a catheter implanted in a subject, the method comprising flushing a lumen of the catheter with a solution comprising alcohol, wherein at least an inner surface of the lumen is impregnated or coated with gentian violet and wherein gentian violet is present in a concentration of 0.01%-20% by weight of the catheter.

42-49. (canceled)

50. The method of claim 41, wherein gentian violet is present in the catheter in a concentration that, in the absence of alcohol, is subinhibitory for attachment or growth of microorganisms on the catheter.

51. The method of claim 41, wherein alcohol is present in a concentration that, in the absence of gentian violet, is subinhibitory for attachment or growth of microorganisms on the catheter.

52. The method of claim 41, wherein gentian violet is present in the catheter in a concentration that, in the absence of alcohol, is subinhibitory for attachment or growth of microorganisms on the catheter, and wherein alcohol is present in a concentration that, in the absence of gentian violet, is subinhibitory for attachment or growth of microorganisms on the catheter.

53. The method of claim 41, wherein alcohol is present in the solution in a concentration of 25-30%.

54. The method of claim 41, wherein growth of Candida albicans, Staphylococcus aureus, or Pseudomonas aeruginosa is inhibited.

55-65. (canceled)

66. The method of claim 41, wherein the catheter is implanted in a blood vessel or a body cavity.

67-69. (canceled)
